(19) United States
(12) Patent Application Publication
(10) Pub. No.: US 2024/0188837 A1
(43) Pub. Date: Jun. 13, 2024

(54) CUFFLESS BLOOD PRESSURE MONITOR WITH MULTIPLE INERTIAL MEASUREMENT UNITS

(71) Applicants: STMICROELECTRONICS, INC., Coppell, TX (US); STMicroelectronics International N.V., Geneva (CH)

(72) Inventors: Mahesh CHOWDHARY, San Jose, CA (US); Vijay KUMAR, Bihar (IN); Goldy, Haryana (IN); Kolin PAUL, New Delhi (IN)

(73) Assignees: STMICROELECTRONICS, INC., Coppell, TX (US); STMicroelectronics International N.V., Geneva, TX (CH)

(21) Appl. No.: 18/063,021

(22) Filed: Dec. 7, 2022

(51) Int. Cl. A61B 5/021; A61B 5/00; A61B 5/026; A61B 5/11

(52) U.S. Cl. CPC A61B 5/021; A61B 5/026; A61B 5/1102; A61B 5/6833; A61B 5/7267; A61B 5/7278; A61B 2562/0219; A61B 2562/043

(57) ABSTRACT

A blood pressure monitoring device includes a patch including two inertial measurement units placed adjacent to the skin of a user. The blood pressure monitoring device includes a control unit coupled to the patch and configured to receive sensor data from the inertial measurement units. The control unit includes an analysis model trained with multiple machine learning processes to generate blood pressure estimations based on the sensor data. A first general machine learning process trains the analysis model with a training set gathered from plurality of other individuals. The second general machine learning process retrains a portion of the analysis model with a second machine learning process utilizing individualized training set gathered from the user.

### BACKGROUND

### Technical Field

The present disclosure is related to blood pressure monitors, and more particularly, to cuffless blood pressure monitors.

### Description of the Related Art

Blood pressure can be a useful metric for individuals in a variety of situations. For example, it can be beneficial for individuals with hypertension to monitor their blood pressure periodically in order to avoid serious medical issues. It can also be beneficial to monitor the blood pressure of individuals when exercising for fitness purposes. It may be beneficial to monitor the blood pressure of individuals for a variety of other reasons.

Typically, blood pressure may be measured at a physician's office. In these cases, a blood pressure monitoring cuff may be wrapped around an individual's arm. The blood pressure cuff includes an inflatable bladder and a hose. The inflatable bladder is inflated to an air pressure sufficient to prevent blood flow in a local main artery. The inflatable bladder is gradually deflated until blood begins to flow through the artery. A pressure sensor measures the pressure at which the blood begins to flow. This corresponds to the systolic blood pressure. The pressure measurement is also taken when blood flow is no longer restricted. This corresponds to the diastolic blood pressure.

While a blood pressure cuff is an effective way of measuring blood pressure, there are several drawbacks. For example, it may be inconvenient or impractical to utilize a blood pressure cuff throughout the day. In particular, a blood pressure cuff is impractical when exercising.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which, in and of itself, may also be inventive.

### BRIEF SUMMARY

Embodiments of the present disclosure provide a cuffless blood pressure monitoring device that can effectively and efficiently monitor the blood pressure of an individual. The blood pressure monitoring device includes one or more sensors that generate sensor signals based, in part, on characteristics of blood flow of the individual. In one embodiment the accelerometer sensor measures the events in the cardiac cycle due to ballistic forces generated by the injection of blood in the cycle. The blood pressure monitoring device includes an analysis model trained with a multiphase machine learning process to estimate the blood pressure of the individual based on the sensor signals. The multiphase machine learning process includes a general machine learning process and an individualized machine learning process. In this way, the analysis model can efficiently and effectively estimate the blood pressure of the individual based on the sensor signals.

In one embodiment, the blood pressure monitoring device includes a first inertial measurement unit and a second inertial measurement unit. The first and second inertial measurement units are positioned in close proximity to the skin of the individual and are spaced apart from each other along a selected direction. The inertial measurement units generate sensor signals based on the blood flow of the individual. The analysis model receives the sensor signals, processes the sensor signals, and generates an estimated blood pressure based on the sensor signals.

In one embodiment, the blood pressure monitoring device is preloaded with an analysis model that has undergone a first machine learning process based on sensor signals and measured blood pressures of a plurality of other individuals. When the user obtains the blood pressure monitoring device, a second machine learning process is performed. During the second machine learning process, a personal training set data is gathered for the user. Gathering the personal training set data includes recording sensor signals generated while the user wears the blood pressure monitoring device. Gathering the personal training set data also includes recording control blood pressure values associated with the periods of time during which the sensor signals are recorded. The second machine learning process is then performed to retrain the analysis model, or a portion of the analysis model, utilizing the personalized training set. The result is that the analysis model can accurately and efficiently predict or estimate the blood pressure of the user based on the sensor signals.

In one embodiment, the analysis model includes a long short-term memory (LSTM) neural network including multiple layers. The first machine learning process may train all layers of the LSTM neural network based on the general training set gathered from the plurality of other individuals. The second machine learning process may retrain one or more layers of the LSTM neural network while one or more other layers of the LSTM neural network are held fixed (i.e., not retrained). The result is a neural network that includes the benefits of general training based on a large number of individuals and the benefits of individualized training based on data gathered from the user.

In one embodiment, the blood pressure monitoring device includes a flexible patch that can be adhered to the skin of the user. The first and second inertial measurement units are embedded in the flexible patch. This dual MEMS accelerometer-based sensor patch can be placed along the length of the carotid artery to monitor the pulsatile motion during the cardiac cycle. The blood pressure monitoring device also includes a control unit coupled to the flexible patch by one or more cables or cords. The first and second inertial measurement units generate sensor signals and provide the sensor signals to the control unit via the one or more cords or cables. The analysis model processes the sensor signals and generates an estimated or predicted blood pressure value based on the sensor signals.

### BRIEF DESCRIPTION OF THE SEVERAL VIEWS OF THE DRAWINGS

Reference will now be made by way of example only to the accompanying drawings. In the drawings, identical reference numbers identify similar elements or acts. In some drawings, however, different reference numbers may be used to indicate the same or similar elements. The sizes and relative positions of elements in the drawings are not necessarily drawn to scale. For example, the shapes of various elements and angles are not necessarily drawn to scale, and some of these elements may be enlarged and positioned to improve drawing legibility.

FIG. 1 is a block diagram of a cuffless one pressure monitor, according to one embodiment.

FIG. 2 is an illustration of a cuffless blood pressure monitor, according to one embodiment.

FIG. 3 is a block diagram of a control unit 106 of the cuffless blood pressure monitor, according to one embodiment.

FIG. 4 is a functional flow diagram illustrating aspects of training an analysis model of a cuffless blood pressure monitor, according to one embodiment.

FIG. 5 is a block diagram of an analysis model with a cuffless blood pressure monitor, according to one embodiment.

FIG. 6 is a graph illustrating sensor signals associated with a cuffless blood pressure monitor, according to one embodiment.

FIG. 7 is a flow diagram of a method for utilizing an analysis model of a cuffless blood pressure monitor, according to one embodiment.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known systems, components, and circuitry associated with integrated circuits have not been shown or described in detail, to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.” Further, the terms “first,” “second,” and similar indicators of sequence are to be construed as interchangeable unless the context clearly dictates otherwise.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its broadest sense, that is as meaning “and/or” unless the content clearly dictates otherwise.

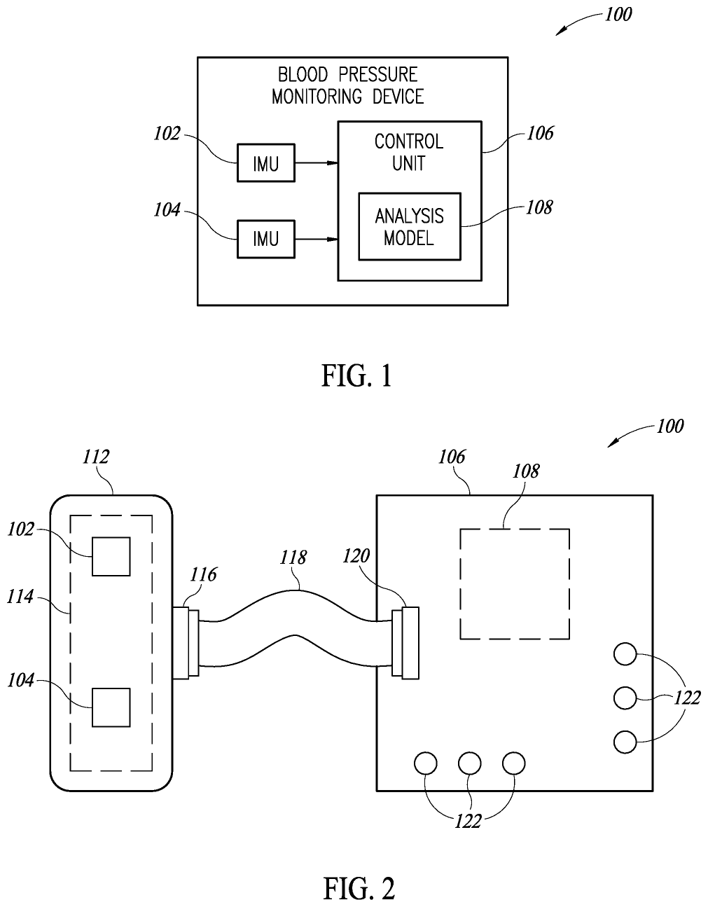

FIG. 1 is a block diagram of a blood pressure monitoring device 100, in accordance with some embodiments. The blood pressure monitor includes a first inertial measurement unit 102, a second inertial measurement unit 104, and a control unit 106. The control unit 106 includes an analysis model 108. As will be set forth in more detail below, the components of the blood pressure monitoring device 100 cooperate to efficiently and effectively estimate the blood pressure of the user of the blood pressure monitoring device 100.

In one embodiment, the blood pressure monitoring device 100 is a cuffless blood pressure monitoring device. Accordingly, the blood pressure monitoring device does not include an inflatable bladder that wraps around an arm of the user. Instead, the blood pressure monitoring device 100 includes at least a portion that is placed adjacent to the skin of the user. For example, a portion of the blood pressure monitoring device may be placed adjacent to a selected artery of the user. The inertial measurement units 102 and 104 are close to the skin of the user. The inertial measurement units 102 and 104 sense vibration or motion of the skin based on the flow of blood through the artery. The inertial measurement units generate sensor signals based on the sensed motion.

The control unit 106 is communicatively coupled to the inertial measurement units 102 and 104. The control unit 106, may be in a housing physically separate from the portion of the blood pressure monitoring device 100 includes the first and second inertial measurement units 102 and 104. For example, a cords or cable may be coupled the first portion of the blood pressure monitoring device 100 to the control unit 106. The control unit 106 may be carried by the user in a pocket or otherwise coupled to the closing of the user. The control unit 106 may also be coupled to a skin of the user via an adhesive, a strap, or in some other manner.

In one embodiment, the control unit 106 is in a same housing as the inertial measurement units 102 and 104. In these cases, the blood pressure monitoring device may include only a single portion coupled to the skin of the user at a selected location. Various configurations of the blood pressure monitoring device 100 can be utilized without departing from the scope of the present disclosure.

In one embodiment, the control unit 106 includes one or more display elements. The one or more display elements can include one or more LEDs. The one or more LEDs can have one or more illumination schemes that indicate whether the blood pressure monitoring device 100 is powered on, whether the blood pressure monitoring device 100 is functioning properly or improperly, whether the estimated blood pressure is in a normal or safe range, whether the estimated blood pressure is in an unsafe range or on the expected range, whether a battery of the control unit 106 is low, or various other types of indications.

In one embodiment, the one or more displays can include a digital display. The digital display can display text or images. The digital display can display the estimated blood pressure, including both systolic blood pressure and diastolic blood pressure. The digital display can display indications of proper function, abnormal function, low power, unsafe blood pressure levels, or other types of information. The digital display can include a liquid crystal display (LCD) or other types of displays.

In one embodiment, the control unit 106 can include one or more user input devices. The user input devices can include buttons, switches, keys, touch screens, or other suitable devices by which a user can input commands to the control unit 106. The input devices can be utilized to determine the blood pressure monitoring device 100 on or off, to select an operating mode of the blood pressure monitoring device, or to perform other functions.

In one embodiment, the inertial measurement unit 102 includes an accelerometer. The accelerometer can include a single axis accelerometer or a multiaxis accelerometers. In one embodiment, the accelerometer is a three axis accelerometer that senses acceleration in three mutually orthogonal axes. The accelerometer can be sensitive to vibrations or motion of the skin resulting from blood flow through the artery adjacent to the inertial measurement unit 102.

In one embodiment, the inertial measurement unit 102 includes a gyroscope. The gyroscope can include a single axis gyroscope or a multiaxis gyroscopes. In one embodiment, the gyroscope is a three axis gyroscope that senses rotation about three mutually orthogonal axes. In one embodiment, the inertial measurement unit 102 includes both an accelerometer and gyroscope.

The inertial measurement unit 104 can include the same types of devices as the inertial measurement unit 102. In particular, the inertial measurement unit 104 can include an accelerometer, a gyroscope, or both of accelerometer and gyroscope. In one embodiment, the inertial measurement unit 104 is substantially identical to the inertial measurement unit 102 except that the inertial measurement unit 104 is separated from the inertial measurement unit 102 by a selected distance. As will be described in more detail below, the selected distance enables the inertial measurement unit 102 to generate sensor signals with a timing offset relative to the inertial measurement unit 104. This can assist in generating an estimation of the blood pressure.

In one embodiment, the inertial measurement units 102 and 104 generates sensor signals. The sensor signals are indicative of accelerations or other movements sensed by the inertial measurement units 102 and 104. As will be described in more detail below, the control unit 106 utilizes the sensor signals to predict or estimate blood pressure.

In one embodiment, the inertial measurement units 102 and 104 generate analog sensor signals and provide the analog sensor signals to the control unit 106. The control unit 106 can include an analog-to-digital converter (ADC) that converts the analog sensor signals into digital sensor data. The digital sensor data can be utilized by the control unit 102 to predict blood pressure.

In one embodiment, the control unit 106 includes digital signal processing (DSP) circuitry that can condition the sensor data generated by the ADC. The DSP may include filters, feature generators, or other circuitry that may condition the digital sensor data prior to the analysis of the digital sensor data to predict or estimate the blood pressure. In one embodiment, the inertial measurement units 102 and 104 can include embedded ADCs to convert the analog sensor signals to digital sensor data prior to providing the digital sensor data to the control unit 106. In one embodiment, the inertial measurement units 102 and 104 can include DSPs for conditioning the digital sensor data prior to providing the digital sensor data to the control unit 106. In one embodiment the inertial measurement units 102 can include digital signal processing blocks and a processor which can execute logic written in high languages (such as C/C++).

The control unit 106 includes an analysis model 108. The analysis model 108 receives the digital sensor data, processes the digital sensor data, and generates a blood pressure prediction or estimation based on the digital sensor data from the inertial measurement units 102 and 104.

The analysis model 108 is trained with a multipart machine learning process. An initial or general machine learning process trains the analysis model with an initial training set that is generated from sensor data and blood pressure measurements of a plurality of individuals that utilized blood pressure monitoring devices similar or identical to the blood pressure monitoring device 100.

The initial training set data may be gathered in the following manner. Each of a plurality of individuals may wear a blood pressure monitoring device including a first and a second inertial measurement unit similar or identical to the inertial measurement units 102 and 104. The inertial measurement units generates sensor data for selected periods of time while the individuals wear the blood pressure monitoring devices. The individuals also utilize a control blood pressure monitor that measures the blood pressure of the individuals during the selected periods of time. The blood pressure measurements are labeled for the training set. Each set of sensor data is matched with a blood pressure measurement. The machine learning process trains the analysis model to accurately predict the blood pressure based on the sets of sensor data. When the predicted blood pressures match the measured blood pressures of the labels within a selected error tolerance the initial machine learning process is complete. This corresponds to pre-training the analysis model 108.

When the blood pressure monitoring device 100 is provided to the user, the pre-trained analysis model 108 is preloaded in the control unit 106. The pre-trained analysis model 108 may be utilized to predict the blood pressure of the individual based on sensor data from the inertial measurement units 102 and 104. At this point, the blood pressure predictions made by the analysis model 108 may be fairly accurate. However, improved accuracy can be achieved by performing a second, individualized machine learning process.

The second machine learning process is an individualized machine learning process for the user of the blood pressure monitoring device 100. After the user has obtained the blood pressure monitoring device 100, the user can control the blood pressure monitoring device 100 to enter an individualized machine learning process. At this point, the user may be prompted to put on both the blood pressure monitoring device 100 and a control blood pressure monitor. During the individualized machine learning process, the control unit 106 records sensor data from the inertial measurement units 102 and 104 over a select period of time. The recorded sensor data makes up part of an individualized training set. The control blood pressure monitor also generates blood pressure measurements during the selected period of time. The blood pressure measurements correspond to labeled data for the individualized training set.

During the machine learning process, the analysis model 108 is retrained utilizing the individualized training set. The analysis model 108 is trained with the individualized training set to work within a threshold error tolerance. In this way, the analysis model 108 is trained to predict or estimate the blood pressure of the individual with high accuracy based on sensor data from the inertial measurement units 102 and 104.

In one embodiment, the analysis model 108 implements a neural network. The neural network includes a plurality of neural layers. The initial or general machine learning process trains each of the layers of the neural network. Training the layers of the neural networks can include identifying weighting values for functions associated with edges or connections between neurons of different neural layers.

In one embodiment, during the second or individualized machine learning process retrains one or more of the layers of the neural network. During the second or individualized machine learning process, one or more other layers of the neural network are held fixed such that functions on weighting values associated with the fixed layers are not retrained during the second or individualized machine learning process. Retraining only a portion of the neural network results in a neural network that has the benefits of the general machine learning process and the individualized machine learning process. After the individualized machine learning process, the analysis model 108 can make blood pressure estimations or predictions with great accuracy for the individual based on the sensor data from the inertial measurement units 102 and 104.

In one embodiment, the neural network is a long short-term memory (LSTM) neural network. The LSTM neural network can include one or more LSTM layers, one or more fully connected layers, and an output layer. The output layer outputs the predicted or estimated blood pressure. Further details regarding LSTM neural networks are provided below. Other types of neural networks and other types of analysis models can be utilized without departing from the scope of the present disclosure.

FIG. 2 is an illustration of a blood pressure monitoring device 100, according to one embodiment. The blood pressure monitoring device 100 of FIG. 2 is one example of a blood pressure monitoring device 100 of FIG. 1.

The blood pressure monitoring device 100 of FIG. 2 includes a patch 112 and a control unit 106. The patch 112 is configured to be placed on the skin of the user at a selected location. In one example, the patch 112 is configured to be placed on the neck of the user over an artery of the user, such as the carotid artery, though other locations can be utilized without departing from the scope of the present disclosure.

The patch 112 may include an adhesive material that adheres the patch 112 to the skin of the user. The adhesive material can include a tape, a glue, or other types of adhesive capable of securely adhering the patch 112 to the skin of the user at the selected location. The patch 112 may be flexible such that the patch 112 can follow or bend with the contours of the skin of the user at the selected location.

The patch 112 includes a circuit board 114. The circuit board 114 may be made from flexible material. The flexible material may enable the circuit board 114 to bend and flex with the contours or shape of the skin of the user.

The patch 112 includes an inertial measurement unit 102 and an inertial measurement unit 104. The inertial measurement units 102 and 104 are coupled to the circuit board 114 of the patch 112. The inertial measurement units 102 and 104 are spaced apart from each other by a selected distance. In the example of the patch 112 being placed over the carotid artery of the user, the carotid artery extends substantially vertically. The patch 112 is oriented on the skin of the user such that the inertial measurement unit 102 is separated from the inertial measurement unit 104 in the vertical direction. More generally, orientation of the patch 112 may be selected based on the orientation of the selected artery or vein over which the patch 112 is placed. The orientation of the patch 112 is selected so that the inertial measurement units 102 and 104 are spaced apart from each other in the direction of travel of the artery or vein.

Estimation of blood pressure using a dual inertial measurement unit patch can be done with the pulse transit time ΔT. Within the same cardiac cycle (pulse), the pulse transit time ΔT is the time for the arterial pulse pressure wave to go from the proximal to the distal position in the same arterial branch. The propagation velocity of a pressure wave through the circulatory system is known as pulse wave velocity (PWV). The relationship between blood pressure (BP) and PWV is represented by the Bramwell-Hills and Moens-Kortweg's equation:

$$PWV = \frac{L}{Δ T} = \sqrt{\frac{hE}{ρ d}} = \sqrt{\frac{{hE}_{0} {e}^{α P}}{ρ d}}$$

where E is the elasticity of arteries, P is the value of blood pressure, ρ is the blood density, d is the artery diameter, h is the artery thickness, α is the Euler number that relies on the condition of the vessels, and Eₒ is Young's modulus for zero arterial pressure elasticity, and L is the separation distance between the two inertial measurement units.

For a small change in ΔT, linearizing the above expression around a nominal ΔT. and assuming all other parameters d, h, L, Eₒ, α, and ρ are constant for an individual, we may obtain the following relationship between BP and ΔT:

BP=a*(ΔT)+b,

where a and b are the subject-specific parameters to be estimated during calibration. The parameters a and b may be found by running a regression between the reference BP and the appropriate time delay ΔT.

One possible approach to computing ΔT is to calculate the difference in time between the arrival times of the sensor signals from the two inertial measurement units. In particular, ΔT can be calculated as the distance between a signal peak S1ₚₑₐₖ from the first inertial measurement unit and a signal peak S2ₚₑₐₖ from the second inertial measurement unit within a heartbeat. In the inertial measurement unit signal, the position of the signal peak corresponds to the maximum ballistic force exerted in the proximal and distal phases of the circularity system. One possible solution is to detect the peak with a DSP. This may utilize a series of signal processing steps to remove high-frequency noises and baseline drift by applying a bandpass filter of the appropriate cutoff frequency. After preprocessing, peak enhancement is performed through amplitude analysis, derivative, digital filtering, matched filter and neural network are used for detection.

These BP estimation solutions may utilize feature engineering through signal processing and rule-based methods. Using this, developing an accurate, personalized model for BP estimation or CVD risk analysis is challenging as the DSP-based methods accuracy varies with age, gender, race, region, demography, body posture, and physical activity.

Accordingly, the blood pressure monitoring device 100 utilizes the machine learning techniques described above to generate an analysis model 108 that can accurately predict the blood pressure of the user based on the sensor data from the inertial measurement units 102 and 104. This results in reduced complexity of the control unit 106, reduced power consumption, and improved blood pressure estimation accuracy.

In one embodiment, the patch 112 includes a first connection port 116. The control unit 106 includes a second connection port 120. A ribbon cable 118 is coupled between the first connection port 116 and the second connection port 120. The ribbon cable 118 communicatively couples the patch 112 to the control unit 106. Sensor signals or sensor data from the inertial measurement units 102 and 104 are passed to the control unit 106 via the ribbon cable 118. Other types of connectors can be utilized to connect the patch 112 to the control unit 106.

In one embodiment, the control unit 106 includes a plurality of user inputs 122. The user inputs 122 can correspond to buttons, switches, sliders, or other types of devices that can be manipulated by the user to control the function of the blood pressure monitoring unit 100. Other types of user inputs can be utilized without departing from the scope of the present disclosure.

In one embodiment, the control unit 106 includes indicator lights 124. The indicator lights 124 can include one or more LEDs. The LEDs can use various illumination schemes to indicate an operational status of the blood pressure monitoring device 100, an indication that blood pressure is within an expected range, an indication that blood pressure is outside of an expected range, that data is being transferred a received, or other types of indications.

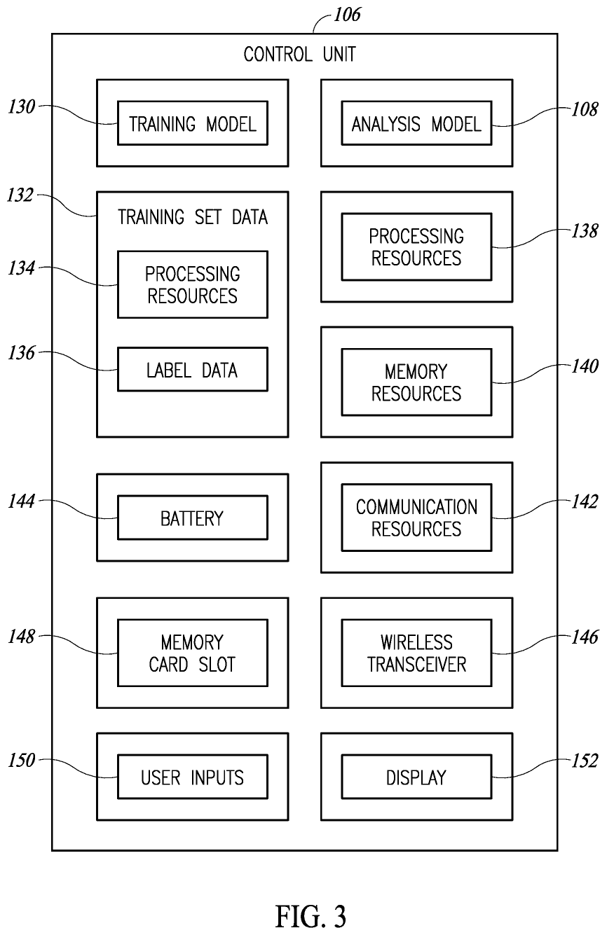

FIG. 3 is a block diagram of the control unit 106, according to one embodiment. The control unit 106 of FIG. 3 is one example of a control unit 106 of FIGS. 1 and 2. The control unit 106 includes an analysis model 108. As described previously, the analysis model 108 is initially preloaded into the control unit 106 prior to use by a user. The analysis model 108 has initially been pre-trained with a general machine learning process. However, as described previously, after the user has received the blood pressure monitoring device 100, a subsequent individualized machine learning process can be performed to further train the analysis model 108.

The control unit 106 includes, or stores, training set data 132. The training set data 132 includes target sensor data 134 and label data 136. The target sensor data 134 includes a plurality of sets of sensor data from the inertial measurement units 102 and 104 while the blood pressure monitoring device 100 is deployed of the user. Each set of previously recorded sensor data corresponds to sensor data collected for a particular period of time. The label data 136 includes a label for each set of sensor data in the target sensor data. Each label corresponds to a control blood pressure measurement taken by the control blood pressure monitor for each set of sensor data.

In one embodiment the analysis model 108 includes a neural network. Retraining of the analysis model 108 will be described in relation to a neural network. However, other types of analysis models or algorithms can be used without departing from the scope of the present disclosure. The retraining process retrains one or more layers of the neural network, while one or more other layers of the neural network are held fixed, as described previously.

In one embodiment, the control unit 106 includes processing resources 138, memory resources 140, and communication resources 142. The processing resources 138 can include one or more controllers or processors. The processing resources 138 are configured to execute software instructions, process data, perform signal processing, read data from memory, write data to memory, and to perform other processing operations.

In one embodiment, the memory resources 140 can include one or more computer readable memories. The memory resources 140 are configured to store software instructions associated with the function of the control unit and its components, including, but not limited to, the analysis model 108. The memory resources 140 can store data associated with the function of the control unit 106 and its components. The data can include the training set data 132, sensor data, and any other data associated with the operation of the control unit 106 or any of its components.

In one embodiment, the communication resources 142 can include resources that enable the control unit 106 to communicate with components associated with the blood pressure monitor device 100 and with external systems. For example, the communication resources 142 can include wired and wireless communication resources that enable the control unit 106 to receive the sensor data and to communicate with external systems. In one embodiment, the analysis model 108 is implemented via the processing resources 138, the memory resources 140, and the communication resources 142.

The control unit 106 may include a battery 144. The battery 144 may correspond to a power source that powers the control unit 106 and the inertial measurement units 102 and 104. The battery 144 may be rechargeable and/or replaceable.

The control unit 106 may include a wireless transceiver 146. The wireless transceiver 146 may be part of the communication resources 142. The wireless transceiver 146 may be utilized to transmit data from the control unit 106 to an external device or system, and to receive data from an external device or system. In one embodiment, the retraining process of the analysis model 108 can be accomplished, in part, with use of the wireless transceiver 146. For example, the wireless transceiver 146 may receive data from the control blood pressure monitor and may provide the control blood pressure data to the control unit 106 during gathering of the individualized training set data. The wireless transceiver 146 can implement Bluetooth standards, NFC standards, Wi-Fi standards, or other types of wireless communication standards.

In one embodiment, retraining of the analysis model 108 is accomplished by the external system. In particular, the wireless transceiver 146 may transmit sensor data to an external system during gathering of the retraining process. The wireless transceiver 146 may also transmit the pre-trained analysis model 108 to an external system. The external system may retrain the analysis model based on the sensor data and control blood pressure values. The external system may then transmit the retrain the analysis model 108 to the control unit 106. The wireless transceiver 146 may facilitate is communication. Alternatively, wired communication may also facilitate communication between the control unit 106 and an external device or system.

The control unit 106 may include a memory card slot 148. The memory card slot 148 may be configured to receive a memory card such as an SD memory card or another type of memory card. The control unit 106 may include other types of slots or ports for receiving other types of memory devices.

The control unit 106 can include user inputs 150. The user inputs 150 can include buttons, switches, sliders, touch screens, input keys, or other types of devices that can enable a user to control functions of the control unit 106.

The control unit 106 can include a display 152. The display 152 can include a display screen, indicator lights, or other types of displays that can output indications or data to the user. In one embodiment, the display 152 can include a screen that displays messages including current estimated or predicted blood pressure values, operational status of the blood pressure monitoring device 100, or other messages or data.

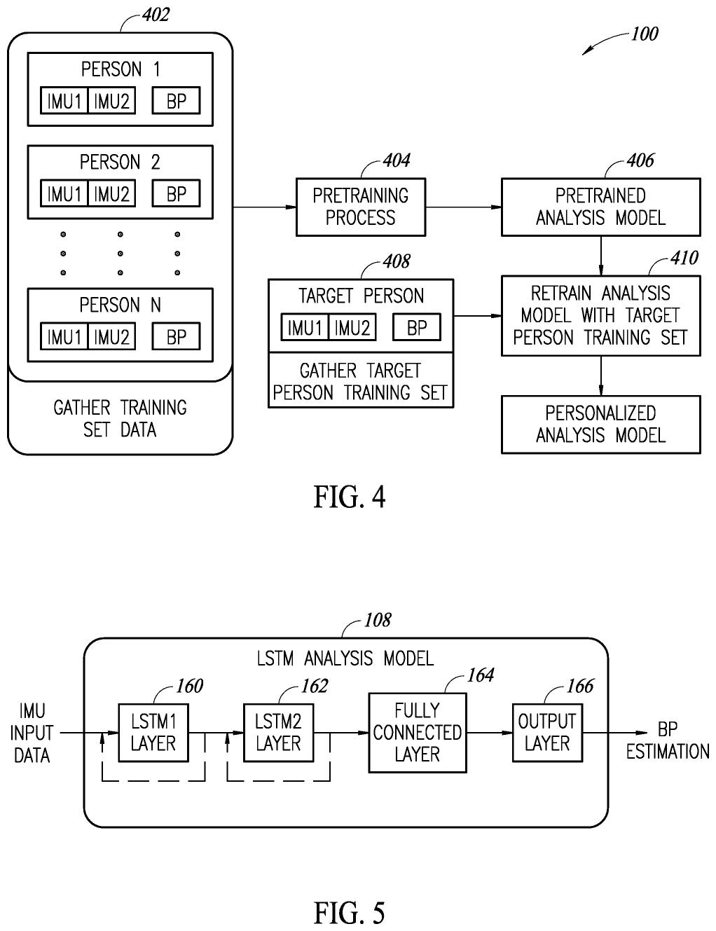

FIG. 4 is a functional flow diagram of a process 400 for training an analysis model 108, according to one embodiment. The process 400 can utilize components, systems, and processes described in relation to FIGS. 1-3. At 402, general training set data is gathered. The general training set data includes sensor data and blood pressure values for a plurality of individuals. In the example of FIG. 4, the general training set data includes sensor data and blood pressure values for n individuals.

At 404, a pre-training process is performed to pretrain the analysis model 108 with the general training set data. The pretraining process is a machine learning process that trains the analysis model 108 to generate predicted or estimated blood pressure values based on sensor data.

At 406, the analysis model 108 has been pre-trained. The pre-trained analysis model can now be loaded into the control unit 106 of a blood pressure monitoring device 100. The blood pressure monitoring device 100 can be utilized by an individual to monitor the individuals blood pressure based on the output of the analysis model 108. However, as described previously, the pre-trained model may not be as accurate as desired because characteristics of the individual that utilizes the blood pressure monitoring device 100 may be different than characteristics in the general training set data. Accordingly, an individualized training set data may be gathered from the individual or target person, as described previously.

At 410, the analysis model 108 is retrained with the individualized training set data. This can include retraining one or more layers of a neural network of the analysis model 108 while holding fixed the values associated with one or more other layers of the neural network.

At 410, the personalized analysis model 108 has been generated by the retraining process. The personalized analysis model 108 can now be utilized to predict or estimated blood pressure values for the user based on sensor data from the inertial measurement units 102 and 104.

FIG. 5 is a block diagram of an analysis model 108, according to one embodiment. In the example of FIG. 5, the analysis model 108 includes an LSTM neural network. The LSTM neural network includes a first LSTM layer 160. The first LSTM layer 160 includes a plurality of neurons. The analysis model 108 may include a second LSTM layer 162. The second LSTM layer 162 includes a plurality of neurons connected to the neurons of the first LSTM layer 160 by edges or weighting values. The second LSTM layer 162 may have a different number of neurons than the first LSTM layer 160. The analysis model 108 includes a fully connected layer 164 downstream from the second LSTM layer 162. The fully connected layer 164 may have a same number of neurons as the second LSTM layer 162. The neurons of the fully connected layer 164 are connected to the neurons of the second LSTM layer 162 by edges or weighting values. The analysis model 108 includes an output layer downstream from the fully connected layer 164.

During operation of the analysis model 108, sensor data from the inertial measurement units 102 and 104 is received at the first LSTM layer 160. Data is the process by the LSTM layer 160 and passed to the second LSTM layer 162. Data is then process by the second layer 162 and passed to the fully connected layer 164. Data is processed by the fully connected layer 164 and passed to the output layer 166. The output layer 166 generates predicted or estimated blood pressure data based on the sensor data.

The LSTM network differs from some neural networks in that the LSTM network has feedback connections, as illustrated by the dashed arrows rerouting data from the output of the first LSTM layer 160 to the input of the first LSTM layer 160, and from the output of the second LSTM layer 162 to the input of the second LSTM layer 162. Accordingly, the LSTM neural network is a type of recurrent neural network that can process sequences of data, such as sequences of data from the inertial measurement units 102 and 104. During training, the connection weights and biases in the network are adjusted once per iteration. Though not shown in FIG. 5, the LSTM neural network may include dropout layers after each of the LSTM layers. Furthermore, the LSTM neural network may include different numbers and configurations of layers than shown in FIG. 5 without departing from the scope of the present disclosure. Other types of neural networks can be utilized for the analysis model 10 a without departing from the scope of the present disclosure.

In one embodiment, the initial or general training process of the analysis model 108 may include training all layers of the LSTM neural network. The retraining or individualized training process may include training values associated with the second LSTM layer 162, the fully connected layer 164, and the output layer 166, while values associated with the first LSTM layer 160 are held constant. In the retraining process, other combinations of layers may be retrained while other combinations of layers remain fixed. In one embodiment, the LSTM may generate blood pressure estimations or predictions for windows of sensor data. In one example, each window of sensor data includes sensor data during a selected period of time. In one example, each window is about five seconds and includes about 4000 samples. Each sample includes six data values, corresponding to each of the three sensing axes of the first and second inertial measurement units 102 and 104. In one example, the first LSTM layer 160 includes 2160 neurons and outputs 20 data values generated from the six data values of the input data. In one example, the second LSTM layer 162 includes 3280 neurons and outputs 20 data values from the 20 data values received from the first LSTM layer 160. In one embodiment, a first dense layer receives 20 data values for sample from the second LSTM layer 162 and outputs 10 data values for sample from 210 neurons. In one embodiment, a second dense layer receives the data values for sample from the first dense layer and outputs two data values from 22 neurons. The two data values output from the output layer correspond to the systolic blood pressure in the diastolic blood pressure. Other numbers of neurons, numbers of layers, and numbers of data values for sample can be utilized without departing from the scope of the present disclosure.

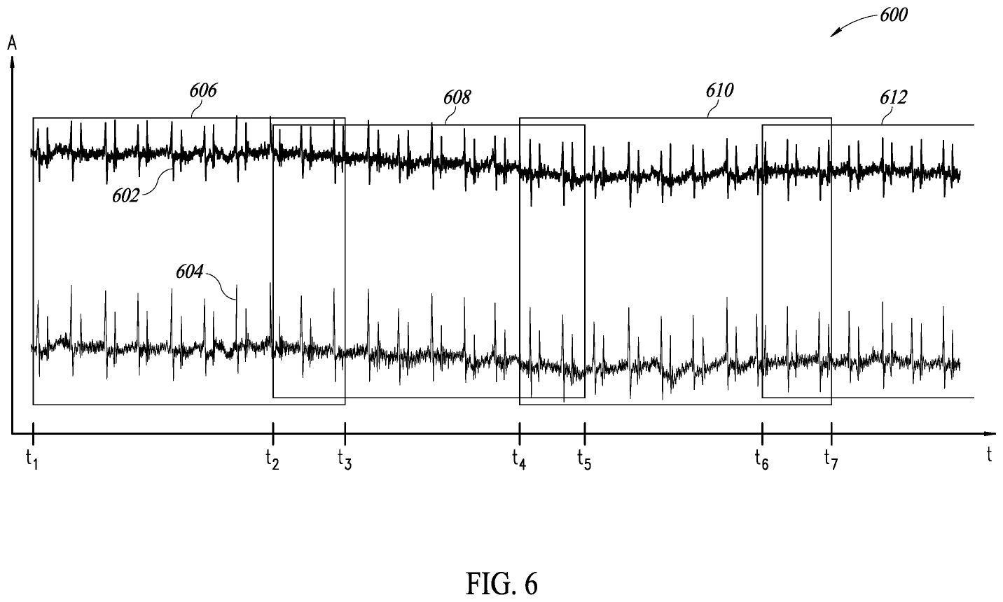

FIG. 6 is a graph 600 illustrating sensor data from the inertial measurement units 102 and 104, according to one embodiment. The graph 600 includes a first curve 602 corresponding to sensor data from the inertial measurement unit 102. The graph 600 includes a second curve 604 corresponding to sensor data from the inertial measurement unit 104. The graph 600 illustrates for timing windows 606, 608, 610, and 612. The first timing window 606 extends from a time T1 to a time T3. The second timing window 608 extends from a time T2 to a time T5. The third timing window 610 extends from the time T4 to the time T7. The fourth timing window 612 begins at a time T6. The end of the time window 612 is not shown in the graph 600.

In one embodiment, each of the timing windows is about 5s long. Each timing window overlaps a previous timing window by about 1s. In one embodiment, the inertial measurement units 102 and 104 sampled at a rate of 833 Hz. This results in about 4000 samples per timing window. In an example in which each inertial measurement units includes a respective three axis accelerometer, each sample includes six data values, one from each axis of the two accelerometers. The analysis model 108 generates an estimated systolic blood pressure in an estimated diastolic blood pressure for each window. Other window durations, overlap values, sampling rates, and data values for sample may be utilized without departing from the scope of the present disclosure.

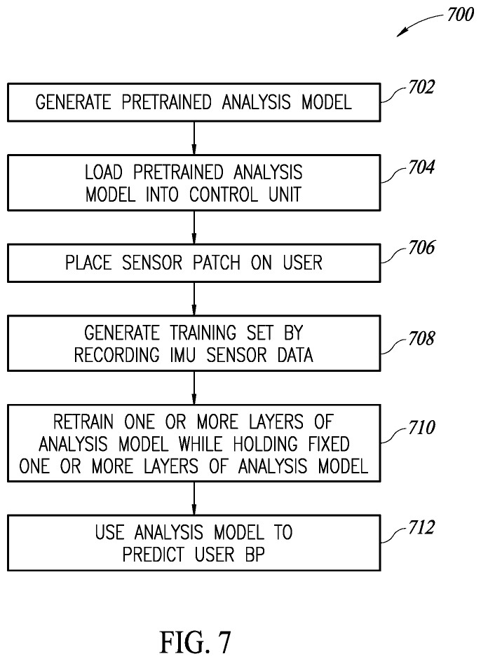

FIG. 7 is a flow diagram of a method 700, according to one embodiment. The process 700 can utilize systems, components, and processes described in relation to FIGS. 1-6. At 702, the method 700 includes generating a pre-trained analysis model. The pre-trained analysis model can be generated by training the analysis model with a machine learning process utilizing a general training set data. At 704, the method 700 includes loading the pre-trained analysis model into the control unit of a blood pressure monitoring device. At 706, a sensor patch of the blood pressure monitoring device is placed on a user. At 708, an individualized training set is generated by recording sensor data from the inertial measurement units. At 710, one or more layers of the analysis model are retrained with a machine learning process utilizing the individualized training set data. At 712, the blood pressure monitoring device utilizes the analysis model to predict or estimate the blood pressure of the user based on sensor data from the inertial measurement units of the blood pressure monitoring device.

In one embodiment, a method includes training an analysis model with a first machine learning process to generate estimated blood pressure values and loading the analysis model into a control unit of a blood pressure monitoring device. The method includes gathering individualized training set data from the blood pressure monitoring device while the blood pressuring monitoring device is coupled to a user and retraining, with a second machine learning process utilizing the individualized training set data, a portion of the analysis model.

In one embodiment, a blood pressure monitoring device includes a patch including a first inertial measurement unit and a second inertial measurement unit each configured to generate sensor data. The blood pressure monitoring device includes a control unit configured to receive the sensor data and including an analysis model trained with a first machine learning process based on general training set data and a second machine learning process based on an individualized training set data generated while a user wears the patch.

In one embodiment, a method includes generating, while a user wears a blood pressure monitoring device, training sensor data. The method includes training a first neural layer of an analysis model of the blood pressure monitoring device with a machine learning process using the training sensor data while holding fixed a second neural layer of the analysis model. The method includes generating, with the analysis model after the machine learning process, estimated blood pressure values for the user.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method, comprising:
training an analysis model with a first machine learning process to generate estimated blood pressure values;
loading the analysis model into a control unit of a blood pressure monitoring device;
gathering individualized training set data from the blood pressure monitoring device while the blood pressuring monitoring device is coupled to a user; and
retraining, with a second machine learning process utilizing the individualized training set data, a portion of the analysis model.

2. The method of claim 1, wherein the blood pressuring monitoring device includes a first inertial measurement unit and a second inertial measurement unit each configured to generate sensor data based on blood flow of the user.

3. The method of claim 2, wherein the blood pressure monitoring device includes a flexible patch configured to be placed on a skin of the user, wherein the first and second inertial measurement units are deployed on the patch.

4. The method of claim 3, wherein gathering the second training set includes receiving blood pressure measurement data from a control blood pressure monitor placed on the user while the blood pressure monitoring device is placed on the user.

5. The method of claim 4, comprising using the blood pressure measurement data as labeled data in the individualized training set data.

6. The method of claim 2, comprising generating, with the analysis model, estimated blood pressure values of the user based on the sensor data after the retraining of the analysis model.

7. The method of claim 2, wherein the analysis model includes a neural network including a first neural layer and a second neural layer, wherein the second machine learning process includes retraining the second neural layer while holding fixed the first neural layer.

8. The method of claim 7, wherein the neural network includes a long short-term memory neural network.

9. The method of claim 8, comprising, after retraining the analysis model:
generating sensor data with the first and second inertial measurement units;
passing the sensor data to the analysis model; and
generating, for each of a plurality of windows of the sensor data, an estimated blood pressure value.

10. The method of claim 9, wherein each window includes a plurality of sensor data samples.

11. The method of claim 9, wherein each estimated blood pressure value includes a systolic blood pressure value and a diastolic blood pressure value.

12. A blood pressure monitoring device, comprising:
a patch including a first inertial measurement unit and a second inertial measurement unit each configured to generate sensor data; and
a control unit configured to receive the sensor data and including an analysis model trained with a first machine learning process based on general training set data and a second machine learning process based on an individualized training set data generated while a user wears the patch.

13. The blood pressure monitoring device of claim 12, wherein the first and second inertial measurement units each include a respective accelerometer.

14. The blood pressure monitoring device of claim 12, wherein the first and second inertial measurement units each include a respective accelerometer and a respective gyroscope.

15. The blood pressure monitoring device of claim 12, wherein the analysis model includes a neural network.

16. The blood pressure monitoring device of claim 15, wherein the neural network is a long short-term neural network.

17. The blood pressure monitoring device of claim 15, wherein the neural network includes a first neural layer and a second neural layer.

18. The blood pressure monitoring device of claim 17, wherein the first machine learning process trains the first neural layer and the second neural layer, wherein the second machine learning process trains the second neural layer but not the first neural layer.

19. A method, comprising:
generating, while a user wears a blood pressure monitoring device, training sensor data;
training a first neural layer of an analysis model of the blood pressure monitoring device with a machine learning process using the training sensor data while holding fixed a second neural layer of the analysis model; and
generating, with the analysis model after the machine learning process, estimated blood pressure values for the user.

20. The method of claim 19, wherein the neural network is a long short-term neural network.

21. The method of claim 19, wherein generating the training sensor data includes generating the training sensor data with a first inertial measurement unit and a second inertial measurement unit of the blood pressure monitoring device.
